(19) United States
(12) Patent Application Publication
(10) Pub. No.: US 2013/0122080 A1
(43) Pub. Date: May 16, 2013

(54) PHARMACEUTICAL COMPOSITION CAPABLE OF IMMUNO-REGULATION OR ENHANCEMENT OF IMMUNE RESPONSE

(71) Applicants: Kuang-Wen LIAO, Hsinchu City (TW); Chia-Hung Chen, Taichung City (TW)

(72) Inventors: Kuang-Wen LIAO, Hsinchu City (TW); Chia-Hung Chen, Taichung City (TW)

(21) Appl. No.: 13/412,430

(22) Filed: Mar. 5, 2012

(30) Foreign Application Priority Data
Nov. 14, 2011 (TW) 100141418

(51) Int. Cl. A61K 9/127; A61K 39/00; A61P 37/04; A61K 39/395

(52) U.S. Cl. 424/450; 424/130.1; 424/173.1; 424/184.1

(57) ABSTRACT

A pharmaceutical composition of immuno-regulation or enhancement of immune response is disclosed, which includes a liposome and an immuno-regulating molecule or an antigenic molecule. The liposome, the immuno-regulating molecule or the antigenic molecule needs no genetic or chemical modification. The immuno-regulating molecule or the antigenic molecule is spontaneously adsorbed by the liposome. Thus, the formed pharmaceutical composition can be used for immuno-regulation or enhancement of immune responses.

### CROSS REFERENCE TO RELATED APPLICATION

This application claims the benefits of the Taiwan Patent Application Serial Number 100141418, filed on Nov. 14, 2011, the subject matter of which is incorporated herein by reference.

### BACKGROUND OF THE INVENTION

1. Field of the Invention

The present invention relates to a pharmaceutical composition containing liposomes, and particularly to a pharmaceutical composition suitable for immuno-regulation or enhancement of immune response.

2. Description of Related Art

Generally, liposomes are vehicles made of phospholipid bilayers and can be employed to achieve drug delivery by encompassing target drug molecules. Because a phospholipid bilayer is a major component in the structure of a cell membrane, it is considered that liposomes commonly have good biocompatibility and bio-metabolizability.

Although application of liposomes as drug vehicles has the aforesaid advantages and features, it is still difficult to achieve industrialization for mass production of liposomes at present. For example, the encapsulation ratio of some water-soluble drugs in liposomes is still low, the drugs easily leak out from liposomes, and the stability of liposomes is poor. Therefore, for the purpose of increasing the stability of liposomes, some chemical modifications are applied in hydrophilic or hydrophobic functional groups of liposomes in order to directly achieve chemical bonding of a drug molecule to liposomes. Nevertheless, additional chemical modifications may cause longer production time or lower activity of liposomes.

Based on the aforementioned, the use and features of liposomes are limited to only encapsulate a drug molecule for drug delivery. Hence, if other methods of liposome encapsulation can be found requiring no chemical modification, the aforesaid problems can be avoided and the applications of liposomes can be further increased.

### SUMMARY OF THE INVENTION

The object of the present invention is to provide a pharmaceutical composition capable of immuno-regulation. In this pharmaceutical composition, various immuno-regulators and liposomes are integrated, and the immuno-regulators naturally adhere to the surface of the liposomes without additional chemical modification for bonding them and also without encapsulating the immuno-regulators in the liposomes. The formed liposome complex thus serves as an artificial antigen-presenting cell and can execute immuno-regulation.

In order to achieve the object, one aspect of the present invention provides a pharmaceutical composition capable of immuno-regulation, including: a liposome and an immuno-regulator which is non-covalently bonded to the liposome. The liposome contains a neutral phospholipid membrane forming a hollow sphere, a positively charged polymer, and an interfacial polymer, wherein the positively charged polymer and the interfacial polymer are non-covalently bonded to the neutral phospholipid membrane, and the ratio of the neutral phospholipid membrane to the positively charged polymer to the interfacial polymer is 10-60:1-3:1-3.

In the mentioned pharmaceutical composition of the present invention, the ratio of the liposome to the immuno-regulator is preferably in a weight range from 1:0.06 to 1:0.15. The kind of immuno-regulator is not particular limited as long as it is a known antibody or protein capable of immuno-regulation and naturally adhering to the surface of the liposome. In one preferred example of the present invention, an anti-CD3 monoclonal antibody (mAb), a combination of an anti-CD3 mAb and an anti-CD28 mAb, and a combination of an anti-CD3 mAb and an anti-CTLA4 mAb are employed as the immuno-regulator.

Another object of the present invention is to provide a pharmaceutical composition capable of enhancing an immune response. In this pharmaceutical composition, various antigenic molecules and liposomes are integrated, and the antigenic molecules naturally adhere to the surface of the liposomes without additional chemical modification for bonding them and also without encapsulating the immuno-regulators in the liposomes. The formed liposome complex is able to induce a cell-medicated immune response in living bodies and will not cause inconsistent immune responses resulting from difficulty in determining the degree of emulsification with a conventional adjuvant.

In order to achieve the object above, another aspect of the present invention provides a pharmaceutical composition for enhancing an immune response, including: a liposome and an antigenic molecule which is non-covalently bonded to the liposome. The liposome contains a neutral phospholipid membrane forming a hollow sphere, a positively charged polymer, and an interfacial polymer, wherein the positively charged polymer and the interfacial polymer are non-covalently bonded to the neutral phospholipid membrane, and the ratio of the neutral phospholipid membrane to the positively charged polymer to the interfacial polymer is 10-60:1-3:1-3.

In the aforementioned pharmaceutical composition of the present invention, the ratio of the liposome to the antigenic molecule is preferably in a weight range from 1.5:1 to 1:1. The kind of antigenic molecule is not particular limited as long as it has antigenicity and is capable of naturally adhering (i.e., non-covalently bonding) to the surface of the liposome. For example, the antigenic molecule can be viruses, proteins, peptides, nucleic acids, oligonucleotides, polysaccharides, polylipids, glycoproteins, lipoproteins, and so on. In a preferred example of the present invention, a heat-shock protein is used as the antigenic molecule.

This pharmaceutical composition can further include an immuno-modulator. Any immuno-modulators commonly known in this field can be used as the immuno-modulator of the present invention. The immuno-modulator serves to enhance relevant immune responses and to promote production of a specific antibody. For example, the immuno-modulator can be cellular/bacterial products such as lipopolysaccharides (LPS), peptidoglycans (PG), lipoteichoic acids (LTA), and lipopeptides/proteins (LP); nucleic acid molecules such as CpG oligodeoxynucleotides (CpG ODN); and cell hormones such as granulocyte-macrophage colony-stimulating factor (GM-CSF). In one preferred example of the present invention, CpG ODN is used as the immuno-modulator to increase the yield of Immunoglobin G2a (IgG2a). In another preferred example, LPS is used as the immuno-modulator to increase the yield of IgA.

In this pharmaceutical composition of the present invention, the immune response can be a Th1 immune response or a cell-mediated immune response.

The capability of immuno-regulation described in the present invention refers to controlling an immune response by inhibiting or activating participators involved in related pathways of the immune response. The participators include factors which influence interaction among T cells, B cells, and macrophages. Besides, the aforesaid enhancement of the immune response refers to activating Th1-immunity and increasing IgG2a more than those with conventional adjuvants. The activation of Th1-immunity and the increase in IgG2a can promote cell-mediated immune responses and increase IgG2a that is twice or more the yield of IgG1. The promotion and the increase are beneficial for defense against pathogens, autoimmune diseases, allergy, and so forth.

The described non-covalent bonding refers to one molecule binding to another molecule by hydrophilic and hydrophobic force, electrostatic force, hydrogen bonds, and Van der Waal force.

In the pharmaceutical compositions of the present invention, the neutral phospholipid membrane contains a phospholipid. The usable phospholipid is not specifically limited as long as the phospholipid bilayer favorably forms in the liposome. For example, the phospholipid can be dilinoleoyl phosphatidyl choline (DLPC), dioleoyl phosphatidyl choline (DOPC), dimyristoyl phosphatidyl choline (DMPC), dipalmitoyl phosphatidyl choline (DPPC), distearoyl phosphatidyl choline (DSPC), dioleoyl phosphatidyl ethanolamine (DOPE), dimyristoyl phosphatidyl ethanolamine (DMPE), dipalmitoyl phosphatidyl ethanolamine (DPPE), dioleoyl phosphatidyl ethanolamine (DOPE), dimyristoyl phosphatidyl phosphatidic acid (DMPA), dipalmitoyl phosphatidic acid (DPPA), dioleoyl phosphatidic acid (DOPA), dimyristoyl phosphatidyl glycerol (DMPG), dipalmitoyl phosphatidyl glycerol (DPPG), dioleoyl phosphatidyl glycerol (DOPG), dimyristoyl phosphatidyl serine (DMPS), dipalmitoyl phosphatidyl serine (DPPS), dioleoyl phosphatidyl serine (DOPS), or a combination thereof. In a preferred example of the present invention, the phospholipid is a combination of DLPC and DOPC.

In the pharmaceutical compositions of the present invention, the positively charged polymer is not particularly limited as long as it can be inserted into the phospholipid bilayer of the liposome and is a polymer having a positive charge. For example, the positively charged polymer can be polyamine, polyethylenimine (PEI), polyvinylpyrrolidone, polylactic acid, or a combination thereof. The positively charged polymer has a weight average molecular weight preferably in a range from 2,500 to 250,000 and more preferably in a range from 5,000 to 125,000. In one preferred example of the present invention, polyethylenimine with a weight average molecular weight from 12,500 to 50,000 is used as the positively charged polymer.

In addition, in the pharmaceutical composition of the present invention, the interfacial polymer is not particularly limited as long as it has both hydrophilic and hydrophobic structures and is uncharged and can be inserted into the phospholipid bilayer of the liposome. For example, the positively charged polymer can be crosslinked polyacrylates, saponin, polyethylene glycol (PEG), or a combination thereof. The interfacial polymer has a weight average molecular weight preferably in a range from 800 to 80,000 and more preferably between 1,600 and 40,000. In one preferred example of the present invention, PEG with a weight average molecular weight between 4,000 and 16,000 is employed as the interfacial polymer.

Accordingly, without chemical modifications for covalent bonding between the liposome and the immuno-regulator, the pharmaceutical composition capable of immuno-regulation according to the present invention can be manufactured within about 20 minutes and is able to function as commendably as acellular or artificial antigen presenting cells (aAPC) that are produced by genetic engineering or chemical modifications. Therefore, the composition will be able to be applied in drug customization to regulate immune responses of animals in the future.

Similarly, without chemical modifications for covalent bonding between the liposome and the antigenic molecule, the pharmaceutical composition capable of enhancing an immune response according to the present invention can be manufactured within about 20 minutes. In this pharmaceutical composition, the antigenic molecule uniformly adheres to the surface of the liposome and there is no need of emulsification. Hence this pharmaceutical composition does not cause difficulty in inconsistent emulsification with conventional commercial adjuvants (complete/incomplete Freund's adjuvant, CFA/IFA) and is able to activate Th1 immunity and to induce or enhance appropriate antibodies of living bodies as well as to improve the yield of a specific type of antibody along with a suitable immuno-modulator.

Other objects, advantages, and novel features of the invention will become more apparent from the following detailed description when taken in conjunction with the accompanying drawings.

### BRIEF DESCRIPTION OF THE DRAWINGS

FIG. 1 is a bar chart of liposome-adsorbing-BSA capacity by BSA added amount in Test Example 1 of the present invention;

FIG. 2 is a bar chart of cell proliferation rate in Test Example 2 of the present invention, wherein a blank is treated with PBS and * means p<0.05 when compared with splenocytes from naive mice; and # represents statistically p<0.05 when compared with liposomes adsorbing anti-CD3 mAb;

FIG. 3 shows bar charts of results of interaction between the immunized cells and the liposome complex in Test Example 3 of the present invention, wherein white bars represent un-immunized cells and black bars represent immunized cells; FIG. 3A shows cell proliferation rate, FIG. 3B shows IL-2 expression, FIG. 3C shows IFN-γ expression, FIG. 3D shows TGF-β expression, and FIG. 3E shows IL-10 expression;

FIG. 4 shows line charts of cell lysis rate in Test Example 4 of the present invention, wherein FIG. 4A represents the addition of un-immunized splenocytes and FIG. 4B represents the addition of splenocytes immunized with an antigen;

FIG. 5 shows the results of induction with Th1-related antibodies in Test Example 5 of the present invention, wherein FIG. 5A is a line chart of antibody induction and FIG. 5B shows ratios of different antibodies; and

FIG. 6 shows the results of induction with immuno-modulators in Test Example 6 of the present invention, wherein FIG. 6A is a line chart of antibody induction and FIG. 6B shows ratios of different antibodies.

### DETAILED DESCRIPTION OF THE PREFERRED EMBODIMENT

In the present invention, the neutral phospholipid membrane forms the phospholipid bilayer of liposomes. The usable phospholipid is a phospholipid commonly used in the instant field and is exemplified as DLPC, DOPC, DMPC, DPPC, DSPC, DOPE, DMPE, DPPE, DOPE, DMPA, DPPA, DOPA, DMPG, DPPG, DOPG, DMPS, DPPS, DOPS, or a combination thereof. Among them, an uncharged phospholipid such as PC is preferred.

The positively charged polymer suitable for the present invention has a weight average molecular weight preferably in a range from 2,500 to 250,000 and more preferably in a range from 5,000 to 125,000. The usable positively charged polymer is exemplified as polyamine, polyethylenimine, polyvinylpyrrolidone, polylactic acid, or a combination thereof.

The interfacial polymer has a weight average molecular weight preferably in a range from 800 to 80,000 and more preferably between 1,600 and 40,000. The usable interfacial polymer is exemplified as crosslinked polyacrylates, saponin, polyethylene glycol (PEG), or a combination thereof.

Hence, when the uncharged phospholipid forms the phospholipid bilayer, the interfacial polymer and the positively charged polymer are able, respectively, to stabilize the structure of the phospholipid bilayer and to impart positive charge thereto.

If the positively charged polymer is polyethylenimine and the interfacial polymer is PEG, the neutral phospholipid membrane, the positively charged polymer, and the interfacial polymer are in a ratio ranging from 10:3:3 to 60:1:1, preferably from 10:3:3 to 30:1:1, and more preferably at 3:1:1.

In the present invention, the component, component rate, and solvent used in the neutral phospholipid membrane are not limited to those described in the preparation example of the present invention. One skilled in the art of the present invention can adjust them if necessary.

Accordingly, the positively charged polymer, the neutral phospholipid membrane, and the interfacial polymer form liposomes in a specific ratio; and the positively charged polymer and the interfacial polymer are distributed on and non-covalently bonded to the neutral phospholipid membrane. In other words, the positively charged polymer and the interfacial polymer can be inserted into the neutral phospholipid membrane owing to their own physical properties. There is no need to chemically bond the polymers to the neutral phospholipid membrane. Therefore, the method for manufacturing liposomes according to the present invention can be achieved by simplified processes and in less time.

In the liposome of the present invention, the positively charged polymer can adsorb a negatively charged substance via electrostatic force. Notably, the liposome has a high capacity for adsorbing substances and the bonding between the liposome and the adsorbed substances is stable and is achieved quickly. Because the liposome non-covalently adsorbs the substances, undesirable influences on or damage to activity or conformation of the adsorbed substances can be prevented.

The pharmaceutical composition of the present invention can be administered topically, vaginally, buccally, parenterally, nasally, through an implanted reservoir, rectally, or by inhalation spray. The term of parenterally means intracranial injection or infusion, intralesional injection, intrathecal injection, intrasternal injection, intrasynovial injection, intraarterial injection, intraarticular injection, intramuscular injection, intravenous injection, intracutaneous injection, or subcutaneous injection techniques.

The pharmaceutical composition of the present invention prepared into, for example, a sterile injectable aqueous or oleaginous suspension can be formulated according to known techniques of this art through using proper wetting or dispersing agents (Tween® 80) and suspending agents. The sterile injectable preparation of the pharmaceutical composition of the present invention can be a solution or suspension. The solution or suspension is prepared in a non-toxic parenterally acceptable diluent or solvent, e.g., 1,3-butanediol. The acceptable vehicles and solvents usable in the present invention can be an isotonic sodium chloride solution, Ringer's solution, water, or mannitol.

Because of the specific embodiments illustrating the practice of the present invention, one skilled in the art can easily understand other advantages and efficiency of the present invention through the content disclosed therein. The present invention can also be practiced or applied by other variant embodiments. Many other possible modifications and variations of any detail in the present specification based on different outlooks and applications can be made without departing from the spirit of the invention.

### Preparation Example 1

### Preparation of Liposomes

First, DLPC and DOPC were taken respectively in an amount of 50 mg and dissolved in 1 mL ethanol to prepare a neutral phospholipid membrane solution. Then, the neutral phospholipid membrane solution was added to a round bottomed flask and evaporated by a rotary evaporator to remove ethanol therein until dry. Thus, a multilayer film-like neutral phospholipid membrane was formed at the bottom of the round bottomed flask.

Subsequently, (0.675 g PEI) and 0.22 g PEG were dissolved in 5 mL deionized distilled water (ddH₂O) to form an aqueous solution containing a positively charged polymer and an interfacial polymer. This aqueous solution was slowly added to the round bottomed flask, of which the neutral phospholipid membrane was formed at the bottom, then gently shaken, violently vibrated for 10 minutes, and finally stood at room temperature overnight. Notably, shaking or vibrating the flask was able to make the neutral lipid membrane transform into a hollow sphere due to hydration (swelling) so that the positively charged polymer and the surface active polymer which were simultaneously hydrophilic and hydrophobic were distributed unevenly on the neutral phospholipid membrane by non-covalent bonding, thereby forming liposomes.

In the present example, in order to get uniform liposomes in the desired size, the formed liposomes further passed through a pore membrane with a pore size of 200 nm, and this passing-through step was repeated nine times.

### Preparation Examples 2-10

### Preparation of Liposome Complexes Adsorbing Immuno-Regulators

The liposomes (10 μg) prepared according to Preparation Example 1 were incubated respectively with anti-CD3 monoclonal antibody (60 ng αCD3 mAb in Example 2), a combination of αCD3 and αCD28 mAbs (both at 60 ng in Example 3), and combinations of αCD3 and αCTLA4 mAbs (60 ng αCD3 and 20 ng αCTLA4 in Example 4; 60 ng αCD3 and 40 ng αCTLA4 in Example 5; 60 ng αCD3 and 60 ng αCTLA4 in Example 6; and 60 ng αCD3 and 120 ng αCTLA4 in Example 7). In addition, the liposomes (100 μg) prepared according to Preparation Example 1 were incubated respectively with a peptide HLA-A2 mononer (0.6 μg in Example 8), a combination of HLA-A2 and αCD28 mAb (0.6 μg HLA-A2 and 0.6 μg αCD28 mAb in Example 9), and a combination of HLA-A2 and αCTLA4 mAb (0.6 μg HLA-A2 and 0.6 μg αCTLA4 mAb in Example 10). After 20 minutes, the cultures were all centrifuged at 5,900×g for 5 minutes. The pellets were resuspended to afford liposome complexes adsorbing different immuno-regulators with different amounts.

### Preparation Examples 11-13

### Preparation of Liposome Complexes Adsorbing Antigenic Molecules

The liposomes (150 μg) prepared according to Preparation Example 1 were incubated respectively with 100 μg HpHsp 60 antigen in Example 11, a combination of 100 μg HpHsp 60 antigen and 2 μg lipopolysaccharide (LPS) in Example 12, and 100 μg HpHsp 60 and 1 μg CpG oligodeoxynucleotide (CpG ODN) in Example 13. After 30 minutes, the cultures were all centrifuged at 5,900×g for 5 minutes. The pellets were resuspended and respectively mixed with 100 μg HpHsp 60 antigen.

### Statistical Analysis

All data of the following tests were analyzed using a SAS statistical software package (SAS Institute, Inc., Cary, N.C.). T-test was used when two independent trials were compared. An ANOVA test was used for comparing multiple variables. Differences of p<0.05 were considered as statistically significant. All the results were expressed as mean±SD.

### Test Example 1

### Protein Uptake Ability

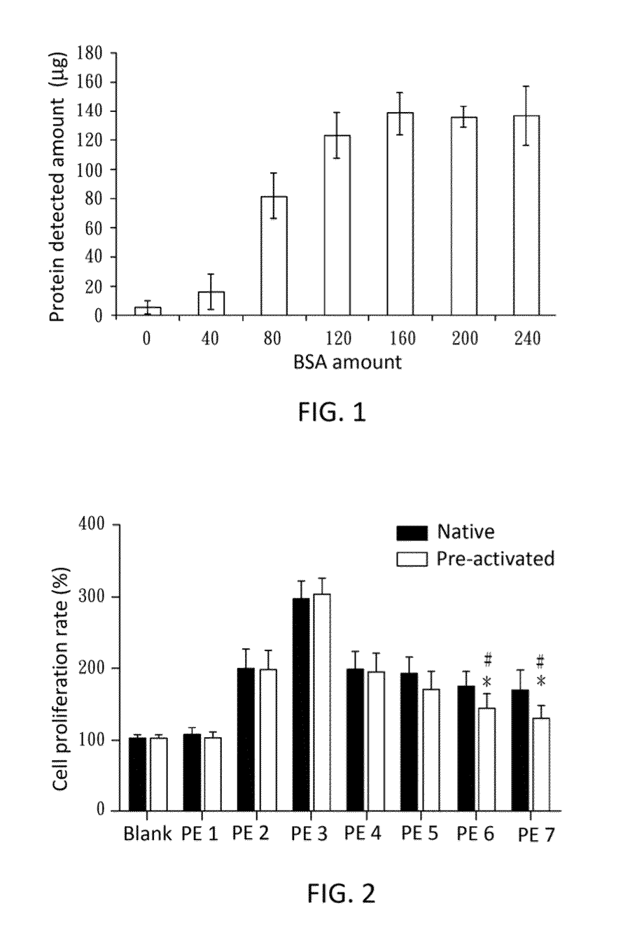

The liposomes (40 μg) prepared according to Preparation Example 1 were incubated respectively with 40 μg, 80 μg, 120 μg, 160 μg, 200 μg, and 240 μg bovine serum albumin (BSA). After 20 minutes, all the cultures were centrifuged at 5,900×g for 5 minutes. The pellets were resuspended and then analyzed by protein quantification for adsorption capacity of the liposomes to BSA. The results are shown in FIG. 1.

With reference to FIG. 1, it can be seen that 40 μg liposomes are able to adsorb at the maximum approximately 140 μg BSA.

### Test Example 2

### Immuno-Activation or Inhibition

The following cell cultures were performed in a 96-well microplate. Each well contained 100 μl RPMI-1640 medium containing 10% fetal calf serium (FCS) and 1% penicillin/streptomycin/amphotericin (PSA).

Splenocytes to be analyzed were classified into a native group and a pre-activated group. In the native group, the splenocytes (2.5×10⁵ cells/well) from 6-8 week old BALB-C mice were incubated respectively with 1 μg liposome complexes of Preparation Examples 2-7 for 3 days.

Additionally, the splenocytes from 6-8 week old BALB-C mice were activated with αCD3 mAb (60 ng/100 μl) and αCD28 mAb (60 ng/100 μl) for 3 days to form those of the pre-activated group. In the pre-activated group, the splenocytes (2.5×10⁵ cells/well) were also incubated respectively with 1 μg liposome complexes of Preparation Examples 2-7 for 3 days.

Then, cytokine (IL-2, IFN-γ, IL-4, IL-10, and TGF-β) profiles were analyzed by ELISA. Splenocyte proliferation rate was monitored by MTT assay. The result is shown in Table 1 and FIG. 2.

TABLE 1

| | IL-2 | IFN-γ | IL-4 | IL-10 | TGF-β |
|---|---|---|---|---|---|
| Blank | 0.18 ± | 1.98 ± | 0.36 ± | 3.14 ± | 0.77 ± |
| | 0.04 | 0.29 | 0.07 | 0.38 | 0.13 |
| Preparation Example 1 | 0.19 ± | 1.90 ± | 0.34 ± | 3.14 ± | 0.70 ± |
| | 0.07 | 0.17 | 0.06 | 0.33 | 0.15 |
| Preparation Example 2 | 0.77 ± | 1.96 ± | 0.45 ± | 2.39 ± | 0.25 ± |
| | 0.07 | 0.14 | 0.04 | 0.17 | 0.05 |
| Preparation Example 3 | 0.96 ± | 2.92 ± | 0.63 ± | 1.78 ± | 0.17 ± |
| | 0.19 | 0.13 | 0.06 | 0.14 | 0.03 |
| Preparation Example 6 | 0.39 ± | 0.65 ± | 0.35 ± | 3.49 ± | 0.86 ± |
| | 0.08 | 0.10 | 0.07 | 0.29 | 0.14 |

Index (pg/ml · %) = (Cytokine conc.)/(Cell proliferation rate)

Table 1 indicates expression of different cytokines (IL-2, IFN-γ, IL-4, IL-10, and TGF-β) after the splenocytes of the native group were treated with the liposome (Preparation Example 1) and the liposome complexes (Preparation Examples 2, 3 and 6) adsorbing different antibodies. The blank means that the splenocytes of the native group were treated with PBS. As shown in FIG. 1, it can be seen that the liposome complexes adsorbing αCD3 mAb, αCD28 mAb, and αCTLA4 mAb are capable of regulating cytokine expression of splenocytes.

In addition, FIG. 2 shows splenocyte proliferation rates analyzed by MTT assay after the splenocytes of the native and pre-activated groups were treated with the liposome complexes adsorbing different antibodies. The blank means that the splenocytes were treated with PBS; * denotes statistically p<0.05 when compared with the splenocytes of the native group; and # represents statistically p<0.05 when compared with liposomes adsorbing αCD3 mAb. As shown in FIG. 2, it is known that the liposome complexes adsorbing αCD3 mAb and/or αCD28 mAb are able to activate splenocyte proliferation, but the liposome complex adsorbing αCTLA4 mAb is able to suppress splenocyte proliferation.

Thus, when the liposomes adsorb both αCD3 and αCTLA4 mAbs, splenocyte proliferation is inhibited more and more obviously with an increase in the amount of αCTLA4 mAbs. When the liposomes adsorb both αCD3 and αCD28 mAbs, splenocyte proliferation doubles and the expression of the cytokines such as IL-2, IL-4, and IFN-γ also increases.

Accordingly, in the pharmaceutical composition of the present invention, the liposomes need no genetic and/or chemical modification. As long as the liposomes naturally adsorb activating immuno-regulators such as αCD3 and/or αCD28 mAbs, non-specific stimulation is able to act on T cells.

### Test Example 3

### Interaction Assay Between Immune Cells and Liposome Complexes

Transgenic mice C57BL/6-Tg (HLA-A2.1) were inoculated with a mixture of 30 μg epitope of human papilloma virus type 16 E7 protein (SEQ ID No. 1: YMLDLQPETT) and Freund's adjuvant. After 3 weeks, splenocytes from these mice were cultured in a 96-well microplate and each well contained about 2.5×10⁵ cells. These splenocytes were classified as an immunized group. In addition, splenocytes from the un-inoculated mice were also cultured in a 96-well microplate and each well contained about 2.5×10⁵ cells. These splenocytes were classified as an un-immunized group.

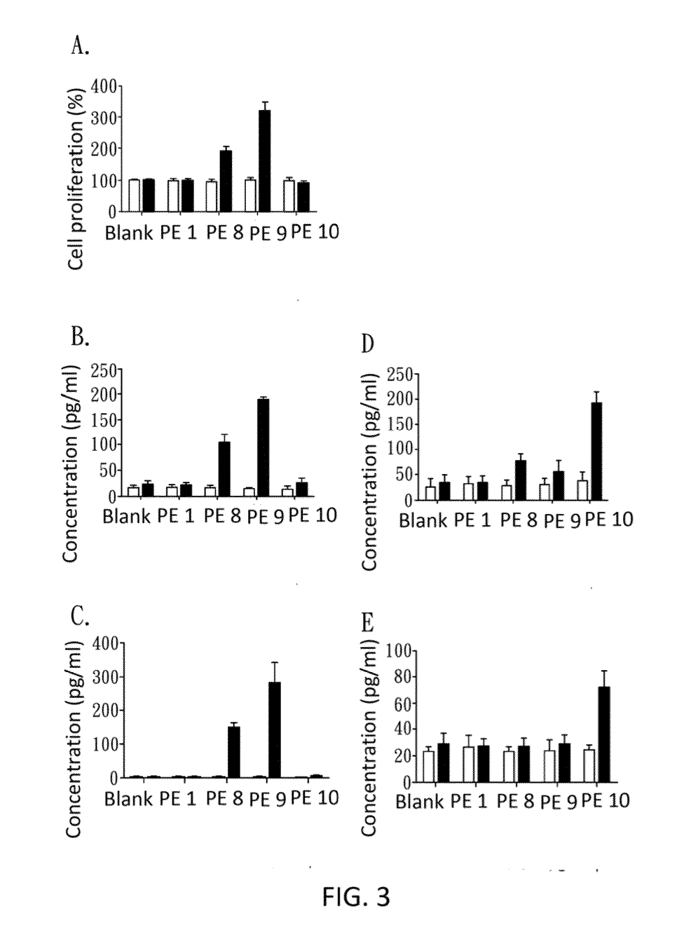

The immunized and un-immunized groups were treated respectively with PBS, the liposome of Preparation Example 1, and the liposome complexes of Preparation Examples 8-10 for 3 days. Then, splenocyte proliferation and cytokine (IL-2, IFN-γ, TGF-β and IL-10) profiles were observed respectively by MTT assay and ELISA. The results are shown in FIGS. 3A-3E. In the figures, white columns denote the un-immunized groups and black columns denote the immunized groups.

With reference to FIG. 3A, it is known that the liposome complexes of Preparation Examples 8 and 9 (Preparation Example 8: liposome+HLA; Preparation Example 9: liposome+HLA+αCD28 mAb) are able to stimulate immunocyte proliferation and the liposome complex of Preparation Example 10 (Preparation Example 10: liposome+HLA+αCTLA4 mAb) is able to efficaciously suppress immunocyte proliferation.

With reference to FIGS. 3B and 3C, IL-2 expression is shown in FIG. 3B and IFN-γ expression is shown in FIG. 3C. As shown in FIGS. 3B and 3C, it is understood that the liposome complexes of Preparation Examples 8 and 9 are able to activate expression of the cytokines such as IL-2 and IFN-γ.

With reference to FIGS. 3D and 3E, TGF-β expression is shown in FIG. 3D and IL-10 expression is shown in FIG. 3E. As shown in FIGS. 3D and 3E, it is understood that the liposome complex of Preparation Example 10 is able to suppress expression of the cytokines such as TGF-β and IL-10.

### Test Example 4

### Cytotoxicity Assay

The transgenic mice were inoculated with PBS (Blank), the liposome of Preparation Example 1, and the liposome complexes of Preparation Examples 8-10. After 2 weeks, the mice were individually boosted by the same materials. Then, one week later, the splenocytes from the inoculated mice were classified as an antigen un-immunized group.

On the other hand, as described in Test Example 3, transgenic mice were inoculated with a mixture of the epitope of human papilloma virus type 16 E7 protein and Freund's adjuvant. After 3 weeks, these mice were further inoculated with PBS (Blank), the liposome of Preparation Example 1, and the liposome complexes of Preparation Examples 8-10. Then, one week later, the splenocytes from these two-step inoculated mice were classified as an antigen immunized group.

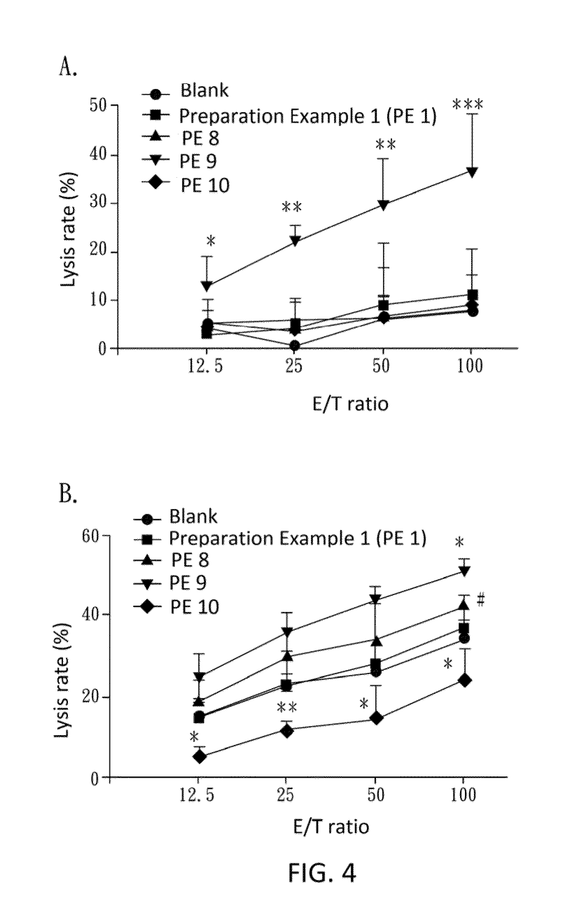

The carcinoma cell line TC1-AAD that expressed the above-mentioned antigen served as target cells and was cultured in a 96-well microplate. Each well contained about 1×10⁴ splenocytes. According to different E/T ratios (12.5/1, 25/1, 50/1, and 100/1), the carcinoma cells were cultured with the splenocytes of the antigen immunized or un-immunized groups. After 72 hours, the lysis rate of the carcinoma cells was analyzed by MTT assay. The results are shown in FIGS. 4A and 4B. The splenocytes of the antigen un-immunized group are shown in FIG. 4A and those of the antigen immunized group are shown in FIG. 4B.

As shown in FIG. 4A, the liposome complex of Preparation Example 9 is able to specifically activate the immunocytes. With the increase in the E/T ratio, the lysis rate of the carcinoma cells also increases. This result is similar to the discoveries described in known papers. In other words, simultaneous provision of two signals (i.e., HLA and αCD28 mAb of Preparation Example 9) is required for specific activation of the immunocytes. Also, as shown in FIG. 4B, the liposome complexes adsorbing different immuno-regulators are able to control the activity of the immunocytes of animals. With the increase in the E/T ratio, the cytotoxic action is actually influenced.

Accordingly, without additional chemical modification and/or purification, the aforesaid liposome complexes adsorbing immuno-regulators are able to act and function as artificial antigen presenting cells (aAPCs).

### Test Example 5

### Induction Assay by Th1-Related Antibodies

BABL/C mice were inoculated with HpHsp 60 antigen (100 μg, Control 1), HpHsp 60 antigen (100 μg) mixed with complete Freund's adjuvant (CFA, 100 μg, Control 2), and the liposome complex of Preparation Example 11. After 2 weeks, the mice were individually boosted by the same materials except CFA of Control 2 was replaced with IFA. Then, one week later, blood from the inoculated mice was collected by eye bleeding once every three days.

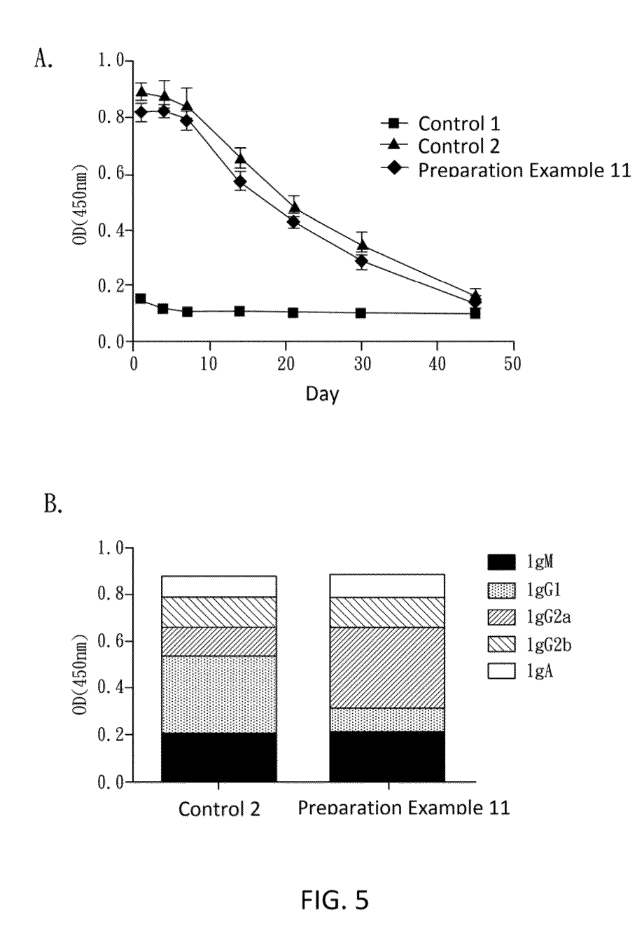

Each well of a 96-well microplate was coated with HpHsp 60 antigen (100 ng), stood at 4° C. overnight, blocked with 300 μl phosphate-buffered saline containing 0.5% Tween-20 (PBST, pH 7.4) and 2% skim milk for 1 hour, and washed once with PBST containing 0.5% skim milk. The collected blood was diluted with phosphate-buffered saline (PBS, pH 7.4) according to blood: PBS=1:800. Subsequently, each well was then cultured with the diluted blood (100 μl) for 2 hours, washed three times, and then incubated with 100 μl HRP-conjugated anti-mouse Ig antibody (1:10,000 dilution) for 1 hour. After being washed three times, each well was incubated with 3,3′,5,5′-tetramethylbenzidine (TMB, 100 μl) for 20 minutes. Finally, 1 N HCl was added to each well to stop reaction, and the optical density of each well measured with an ELISA reader at 450 nm. The results are shown in FIGS. 5A and 5B.

As shown in FIG. 5A, the liposome complex of Preparation Example 11 is actually able to induce antibody (to HpHsp 60) production in the mice when compared with Control 1 (no adjuvant) and exhibit analogous performance with Control 2 (using both of IFA and CFA).

Additionally, as shown in FIG. 5B, the liposome complex of Preparation Example 11 induces more IgG2a (IgG2a/IgG1>1) when compared with Control 2 (using both of IFA and CFA). This result indicates the immune response induced by the liposome complex is classified into Th1-mediated response and is different from that induced by Control 2 (using both of IFA and CFA).

### Test Example 6

### Induction Assay when Incorporated with Immuno-Modulators

Based on the details of Test Example 5, Balb/c mice were inoculated by subcutaneous (s.c.) injection respectively with HpHsp 60 antigen (100 μg) mixed with complete Freund's adjuvant (CFA, 100 μg) (Control 2) and the liposome complexes of Preparation Examples 12 and 13. After 2 weeks, the mice were individually boosted by the same materials except CFA of Control 2 was replaced with IFA. Then, one week later, blood from the inoculated mice was collected by eye bleeding once every three days.

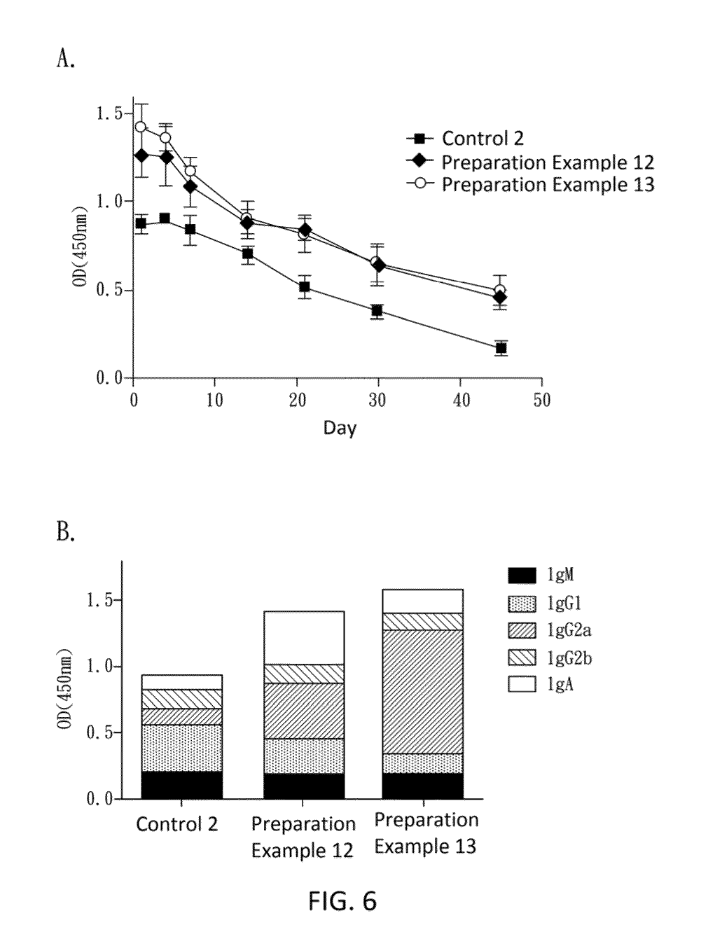

Then, with reference to the steps of Test Example 5, each well of a 96-well microplate was coated with HpHsp 60 antigen (100 ng), stood at 4° C. overnight, blocked with 300 μl phosphate-buffered saline containing 0.5% Tween-20 (PBST, pH 7.4) and 2% skim milk for 1 hour, and washed once with PBST containing 0.5% skim milk. The collected blood was diluted with phosphate-buffered saline (PBS, pH 7.4) according to blood: PBS=1:800. Subsequently, each well was then cultured with the diluted blood (100 μl) for 2 hours, washed three times, and then incubated with 100 μl HRP-conjugated anti-mouse Ig antibody (1:10,000 dilution) for 1 hour. After being washed three times, each well was incubated with 3,3′,5,5′-tetramethylbenzidine (TMB, 100 μl) for 20 minutes. Finally, 1 N HCl was added to each well to stop reaction, and the optical density of each well measured with an ELISA reader at 450 nm. The results are shown in FIGS. 6A and 6B.

As shown in FIG. 6A, the liposome complexes of Preparation Examples 12 and 13, which adsorb the immuno-modulators, exhibit better performance on induction of immune responses than that of Control 2 (using the adjuvants without the immuno-modulators). Additionally, as shown in FIG. 6B, when compared with Control 2 (using the adjuvants without the immuno-modulators), the liposome complexes of Preparation Examples 12 and 13, which respectively adsorb LPS and CpG ODN, are actually able to enhance IgA and IgG2a production, respectively. Accordingly, it is evidenced that the pharmaceutical composition of the present invention is able to control the yield of different antibodies when incorporated with different immuno-modulators.

Although the present invention has been explained in relation to its preferred embodiment, it is to be understood that many other possible modifications and variations can be made without departing from the spirit and scope of the invention as hereinafter claimed.

What is claimed is:

1. A pharmaceutical composition capable of immuno-regulation, comprising:
a liposome containing a neutral phospholipid membrane forming a hollow sphere, a positively charged polymer, and an interfacial polymer, wherein the positively charged polymer and the interfacial polymer are non-covalently bonded to the neutral phospholipid membrane, and the ratio of the neutral phospholipid membrane to the positively charged polymer to the interfacial polymer is 10-60:1-3:1-3, and
an immuno-regulator which is non-covalently bonded to the liposome.

2. The pharmaceutical composition of claim 1, wherein the positively charged polymer is at least one selected from a group consisting of polyamine, polyethylenimine (PEI), polyvinylpyrrolidone, and polylactic acid.

3. The pharmaceutical composition of claim 2, wherein the interfacial polymer is at least one selected from a group consisting of crosslinked polyacrylate, saponin, and polyethylene glycol (PEG).

4. The pharmaceutical composition of claim 3, wherein the neutral phospholipid membrane contains a phospholipid which is at least one selected from a group consisting of dilinoleoyl phosphatidyl choline (DLPC), dioleoyl phosphatidyl choline (DOPC), dimyristoyl phosphatidyl choline (DMPC), dipalmitoyl phosphatidyl choline (DPPC), distearoyl phosphatidyl choline (DSPC), dioleoyl phosphatidyl ethanolamine (DOPE), dimyristoyl phosphatidyl ethanolamine (DMPE), dipalmitoyl phosphatidyl ethanolamine (DPPE), dioleoyl phosphatidyl ethanolamine (DOPE), dimyristoyl phosphatidyl phosphatidic acid (DMPA), dipalmitoyl phosphatidic acid (DPPA), dioleoyl phosphatidic acid (DOPA), dimyristoyl phosphatidyl glycerol (DMPG), dipalmitoyl phosphatidyl glycerol (DPPG), dioleoyl phosphatidyl glycerol (DOPG), dimyristoyl phosphatidyl serine (DMPS), dipalmitoyl phosphatidyl serine (DPPS), and dioleoyl phosphatidyl serine (DOPS).

5. The pharmaceutical composition of claim 4, wherein the phospholipid is a combination of DLPC and DOPC.

6. The pharmaceutical composition of claim 1, wherein the immuno-regulator is an antibody.

7. The pharmaceutical composition of claim 6, wherein the antibody is an anti-CD3 antibody, an anti-CD28 antibody, an anti-CTLA4 antibody or a combination thereof.

8. The pharmaceutical composition of claim 1, wherein the ratio of the liposome to the immuno-regulator is in a weight range from 1:0.06 to 1:0.15.

9. A pharmaceutical composition for enhancing an immune response, comprising:
a liposome containing a neutral phospholipid membrane forming a hollow sphere, a positively charged polymer, and an interfacial polymer, wherein the positively charged polymer and the interfacial polymer are non-covalently bonded to the neutral phospholipid membrane, and the ratio of the neutral phospholipid membrane to the positively charged polymer to the interfacial polymer is 10-60:1-3:1-3, and
an antigenic molecule which is non-covalently bonded to the liposome.

10. The pharmaceutical composition of claim 9, wherein the positively charged polymer is at least one selected from a group consisting of polyamine, polyethylenimine (PEI), polyvinylpyrrolidone, and polylactic acid.

11. The pharmaceutical composition of claim 10, wherein the interfacial polymer is at least one selected from a group consisting of crosslinked polyacrylate, saponin, and polyethylene glycol (PEG).

12. The pharmaceutical composition of claim 11, wherein the neutral phospholipid membrane contains a phospholipid which is at least one selected from a group consisting of dilinoleoyl phosphatidyl choline (DLPC), dioleoyl phosphatidyl choline (DOPC), dimyristoyl phosphatidyl choline (DMPC), dipalmitoyl phosphatidyl choline (DPPC), distearoyl phosphatidyl choline (DSPC), dioleoyl phosphatidyl ethanolamine (DOPE), dimyristoyl phosphatidyl ethanolamine (DMPE), dipalmitoyl phosphatidyl ethanolamine (DPPE), dioleoyl phosphatidyl ethanolamine (DOPE), dimyristoyl phosphatidyl phosphatidic acid (DMPA), dipalmitoyl phosphatidic acid (DPPA), dioleoyl phosphatidic acid (DOPA), dimyristoyl phosphatidyl glycerol (DMPG), dipalmitoyl phosphatidyl glycerol (DPPG), dioleoyl phosphatidyl glycerol (DOPG), dimyristoyl phosphatidyl serine (DMPS), dipalmitoyl phosphatidyl serine (DPPS), and dioleoyl phosphatidyl serine (DOPS).

13. The pharmaceutical composition of claim 12, wherein the phospholipid is a combination of DLPC and DOPC.

14. The pharmaceutical composition of claim 9, wherein the antigenic molecule is a heat-shock protein.

15. The pharmaceutical composition of claim 14, wherein the ratio of the liposome to the antigenic molecule is in a weight range from 1.5:1 to 1:1.

16. The pharmaceutical composition of claim 9, further comprising: an immuno-modulator.

17. The pharmaceutical composition of claim 16, wherein the immuno-modulator is an oligodeoxynucleotide, a lipopolysaccharide, or a combination thereof.

18. The pharmaceutical composition of claim 9, wherein the immune response is Th1 immune response.

19. The pharmaceutical composition of claim 18, wherein the immune response is a cell-mediated immune response.


SEQUENCE LISTING (1 sequence)

SEQ ID NO: 1 - protein, 10 aa, Human papillomavirus type 16
YMLDLQPETT